(19) United States
(12) Patent Application Publication
(10) Pub. No.: US 2023/0405401 A1
(43) Pub. Date: Dec. 21, 2023

(54) MUSICAL INSTRUMENT THERAPY DEVICE

(71) Applicant: Jerry Chi HU, Soldotna, AK (US)

(72) Inventors: Jerry Chi HU, Soldotna, AK (US); Brenton FINLAYSON, Highett, Victoria (AU)

(73) Assignee: Jerry Chi Hu, Soldotna, AK (US)

(21) Appl. No.: 18/250,688

(22) PCT Filed: Sep. 17, 2021
(86) PCT No.: PCT/US2021/050927

§ 371 Date: Apr. 26, 2023

Related U.S. Application Data
(60) Provisional application No. 63/106,134, filed on Oct. 27, 2020.

(51) Int. Cl. A63B 23/18

(52) U.S. Cl. CPC A63B 23/185; A63B 2220/56

(57) ABSTRACT

A musical instrument therapy device for providing strengthening therapy for sleep apnea treatment. Utilization of the device can help to strengthen the user's muscles of the throat and tongue, which can help the user to maintain an open airway during sleep (reduce airway collapse) and prevent airway blockages, thereby reducing the chance of obstructed breathing during sleep. The musical instrument therapy device comprises: a tubular body defining an air column; a mouthpiece located at a first end of the tubular body configured to receive a blow input; a mute located at a second end of the tubular body configured to mute the blow input; a plurality of sensors configured to obtain measurements associated with the blow input; a processing unit configured to make one or more determinations associated with blow input based on the obtained measurements; and an indicator configured to display an indication of the one or more determinations.

### CROSS-REFERENCE TO RELATED APPLICATIONS

The present application is a PCT International Patent Application and claims the benefit of U.S. Provisional Patent Application No. 63/106,134, titled “Musical Instrument Therapy Device,” filed Oct. 27, 2020, the entire disclosure of which is hereby incorporated by reference in its entirety.

### BACKGROUND

Sleep disorders, such as obstructive sleep apnea (OSA), are prevalent amongst the general population. If untreated, sleep disorders can increase the risk of various health problems, such as obesity, diabetes, cardiovascular disease, and depression. For example, sleep apnea is a condition in which the airway between the nose and lungs is blocked, causing interruption of sleep and low energy levels throughout the day. People diagnosed with sleep apnea are often prescribed a range of therapy or treatment options, such as CPAP (Continuous Positive Airway Pressure) devices, oral appliance devices, and surgeries. As can be appreciated, CPAP devices and other therapy or treatment options oftentimes have low rates of adherence. Oftentimes, this can be due in part to uncomfortable masks, claustrophobic sensations, negative side effects such as skin irritation and nasal stuffiness, etc.

An alternative to surgery or treatment devices is natural strengthening therapy that treats sleep apnea at the source of the condition. According to an aspect, various studies have shown a correlation between playing some wind instruments, such as a didgeridoo, a drone instrument traditional among Australian Aborigines, and improvements in breathing during sleep and lowered incidence of fatigue during the day. For example, playing some wind instruments, such as a didgeridoo, can strengthen a player's muscles in his/her windpipe. By strengthening the muscles of the throat and tongue, wind instrument playing helps to maintain an open airway during sleep (helps to reduce airway collapse) and prevent airway blockages, thereby reducing the chance of obstructed breathing during sleep. While wind instruments, such as didgeridoos, can be utilized to strengthen the player's muscles of the throat and tongue, they can also be noisy. As can be appreciated, the noise, the size, and/or other reasons may detract those who may experience sleep apnea from playing the didgeridoo for sleep apnea therapy.

Accordingly, a therapy device is needed for providing a non-surgical and natural therapy that treats sleep apnea at the source of the condition. Also, while relatively specific problems have been discussed, it should be understood that the embodiments should not be limited to solving the specific problems identified in the background.

### SUMMARY

The disclosure generally relates to a musical instrument therapy device for providing throat muscle strengthening therapy or training. For example, the present disclosure describes the musical instrument and a method for providing throat muscle strengthening therapy, which can be used for providing treatment of sleep apnea, training or therapy in association with playing an actual wind instrument, etc.

In one aspect, a musical instrument therapy device is provided, comprising: a tubular body defining an air column; a mouthpiece located at a first end of the tubular body configured to receive a blow input; a mute located at a second end of the tubular body configured to mute the blow input; at least one sensor configured to obtain measurements associated with the blow input; and an indicator configured to provide an indication of the measurements.

In another aspect, a system is provided, comprising: at least one processing device; a memory that stores computer executable instructions for providing strengthening therapy, which when executed by the at least processing device, operates to: receive, from a musical instrument therapy device, measurements obtained by at least one sensor in the musical instrument therapy device, wherein the measurements are associated with a blow input received by the musical instrument therapy device; and provide an indication of the received measurements.

In another aspect, a method is described for providing strengthening therapy comprising: receiving a blow input via a mouthpiece of a musical instrument therapy device; obtaining one or more measurements associated with the blow input from at least one sensor; and providing an indication of the one or more measurements. This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following figures:

FIG. 1 is a is a perspective illustration of an example musical instrument therapy device according to an embodiment;

FIG. 2 is an illustration of a player utilizing a circular breathing technique to operate an example musical therapy device according to an embodiment;

FIG. 3 is a flow diagram depicting general stages of an example process for providing strengthening therapy;

FIG. 4 is a flow diagram depicting general stages of an example process for providing circular breathing support; and

FIG. 5 is a block diagram illustrating example physical components of a computing device or system with which embodiments may be practiced.

### DETAILED DESCRIPTION

The following detailed description refers to the accompanying drawings. Wherever possible, the same reference numbers are used in the drawings and the following description to refer to the same or similar elements. While aspects of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the present disclosure, but instead, the proper scope of the present disclosure is defined by the appended claims. The following detailed description is, therefore, not to be taken in a limiting sense.

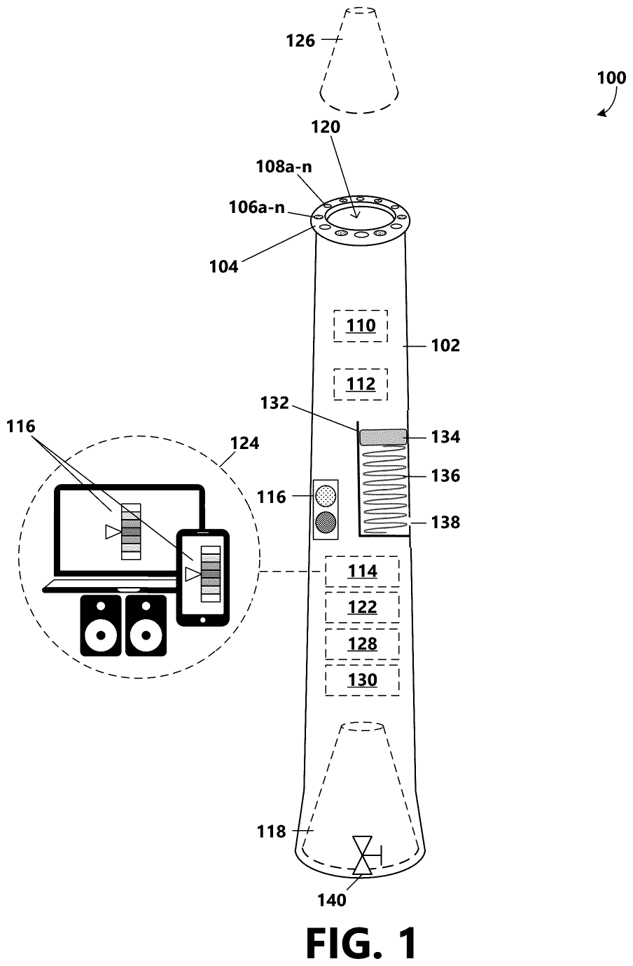

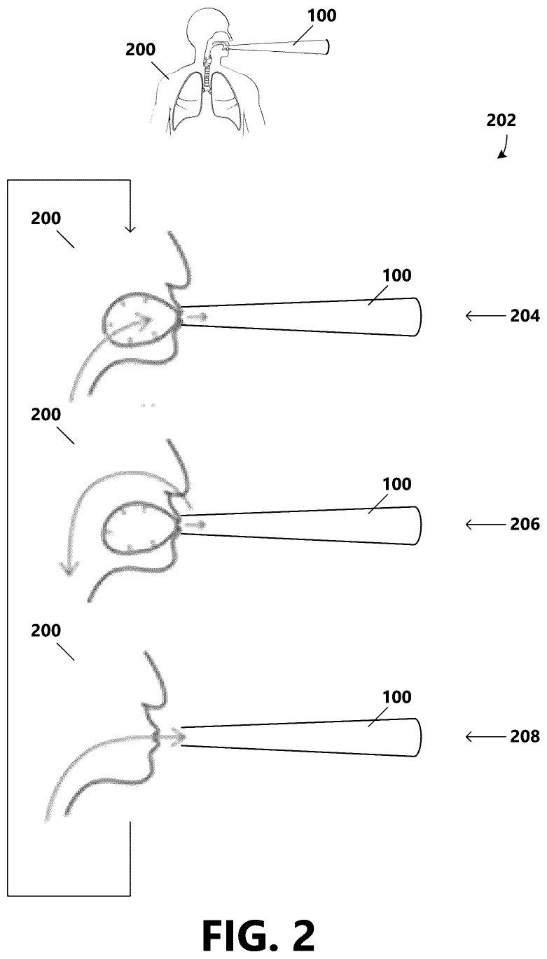

The present disclosure provides a musical instrument therapy device for providing throat muscle strengthening therapy or training. Example illustrations of an example musical instrument therapy device 100 are provided in FIGS. 1-2. In one example implementation, the musical instrument therapy device 100 may be designed and configured to represent a drone instrument, such as a didgeridoo. For example, the musical instrument therapy device 100 may appear similar to a drone instrument and/or function to receive a blow input similarly to a drone instrument. In some examples, the musical instrument therapy device 100 may be adapted (e.g., via an interchangeable mouthpiece) to provide therapy or training in association with other types of musical wind instruments (e.g., trumpet, French horn, English horn).

Various studies have shown a correlation between playing a musical instrument, such as a didgeridoo, a drone instrument traditional among Australian Aborigines, or another wind instrument, and improvements in breathing during sleep and lowered incidence of fatigue during the day. For example, playing a drone instrument (or other wind instrument) can activate and strengthen a player's muscles and tissue in the player's windpipe, cheeks, and/or tongue. By strengthening these muscles and tissue, playing the drone instrument or other wind instrument can help the player to maintain an open airway during sleep and prevent airway blockages, thereby reducing the chance of obstructed breathing during sleep.

Traditionally, a didgeridoo may be made of hollow wood, such as a small tree trunk whose core has been eaten by termites. The player may use the didgeridoo to create an output sound by placing his/her lips on one end of the didgeridoo and blowing into it with loose lips, creating a vibration that echoes down the tube exiting out amplified as a drone. As can be appreciated, people who want to train their throat, cheek, and/or tongue muscles or who experience sleep apnea may wish to play a didgeridoo (or other wind instrument) to train or help treat their sleep apnea; however, because of the loud drone sound of the instrument, which may be an annoyance to others in proximity, and/or for various other reasons (e.g., size or weight of the instrument), some people may not want to play an actual instrument.

When a person wants to receive therapeutic benefit of playing a wind instrument but does not want to play the actual instrument, the person may utilize the musical instrument therapy device 100 for strengthening therapy for sleep apnea treatment, circular breathing training, other musical instrument training, etc. In one example aspect and as illustrated in FIG. 1, the musical instrument therapy device 100 comprises a generally tubular body 102, a mouthpiece 104, a sensor system comprising at least one sensor, and a mute 118. In some examples, the musical instrument therapy device 100 further comprises an indicator 116 to provide results/indications of measurements relating to a player's utilization of the musical instrument therapy device 100.

In some examples, the musical instrument therapy device 100 further comprises a backpressure regulator 140 that may operate to adjust the amount of backpressure in the musical instrument therapy device 100. The backpressure regulator 140, for example, may include an adjustment mechanism that may be adjusted by the user of the musical instrument therapy device 100, wherein adjustment of the adjustment mechanism may cause the backpressure in the musical instrument therapy device 100 to increase or reduce the amount of pressure required to perform a blow input. In some examples, the backpressure regulator 140 may be integrated in the mute 118. In other examples, the backpressure regulator 140 may be a separate component.

In some examples, the musical instrument therapy device 100 may further comprise a bellow 132, which may operate to control an internal pressure in the therapy device 100 and/or a flowrate exiting the therapy device 100. For example, the bellow 132 may operate to help support and/or train the player to perform a circular breathing technique. For example, circular breathing is a technique used by drone instrument players and musicians of other types of wind instruments to produce a continuous sound by occasionally breathing in through the nose while simultaneously pushing air out through the mouth using air stored in the cheeks.

An example circular breathing technique or process 202 that may be performed by a musical instrument therapy device player 200 to utilize the musical instrument therapy device 100 is illustrated in FIG. 2. In one example, the circular breathing process 202 may be performed by the player 200 to help strengthen the pharyngeal airway muscles to help to maintain an open airway during sleep and prevent airway blockages, thereby reducing the chance of obstructed breathing during sleep.

At a first step 204 in the circular breathing process 202, the player 200 may, while providing a blow input into the musical instrument therapy device 100 via the mouthpiece 104, push air out from the lungs and fill the mouth (e.g., puff cheeks with air from the lungs). At a next step 206 in the circular breathing process 202, the player 200 may tighten muscles in the throat to separate the mouth air from the lung air, and quickly inhale air into the lungs through the nose to replenish the lungs while continuing the blow input by expelling the air in the mouth by using the cheek muscles. This maintains the blow input. The circular breathing process 202 may then be repeated as the player 200 returns to step 204 to continue the blow input by re-opening the throat muscles and pushing air into the musical instrument therapy device 100 with the lungs.

Using the circular breathing process 202, the player 200 may be enabled to provide a continuous blow input for a desired time period. As can be appreciated, performing or learning the circular breathing process 202 can sometimes be challenging. According to an example implementation, the bellow 132 may help make circular breathing easier to perform, which may help bridge the gap between an unskilled player 200 and a player 200 who may be able to perform the circular breathing process 202 unassisted.

In some examples and with reference again to FIG. 1, the bellow 132 may be configured as a spring-loaded mechanism. For example, the bellow 132 may define a vent hole 138, and may include a plunger 134 in contact with a spring 136. The bellow 132 may be configured to move downward corresponding to a blow input received from the player 200, wherein the spring 136 may apply a spring-force upward against the plunger 134. The amount of spring-force exerted by the spring 136 may be adjustable, such a via a screw mechanism. For example, as the player 200 provides (204) a blow input into the musical instrument therapy device 100 via the mouthpiece 104 by pushing air out from the lungs, the bellow 132 may receive a portion of the blow input, causing the plunger 134 to move downward against the spring 136 and some air to be expelled through the vent hole 138.

As the player 200 attempts to perform the next step 206 in the circular breathing process 202, the blow input may be maintained at least in part by the bellow 132. For example, when trying to learn to perform the circular breathing technique 202, air pressure of the player's blow input expelled from the player's mouth using the cheek muscles may be less than the spring-force exerted by the spring 136. Accordingly, this may cause the plunger 134 to move upward and cause the portion of blow input received by the bellow 132 to be pushed through the musical instrument therapy device 100. Thus, the bellow 132 may help to maintain the blow input at a generally uniform flowrate. For example, this may allow the player 200 to have additional time to inhale air into the lungs through the nose to replenish the lungs to repeat the process 202. In some examples, the vent hole 138 may vary in size and/or may be covered or uncovered by the player to dampen or disable the bellow 132. In other examples, other types of bellow mechanisms (e.g., a balloon mechanism) may be utilized to maintain internal air pressure or the expelled air flow rate.

Utilization of the musical instrument therapy device 100 can further assist in training a player 200 to play any musical wind instrument by exercising the muscles and airway without the typical sound, music, or noise associated with playing a musical wind instrument.

In some examples, the circular breathing process 202 may be evaluated based on signals collected from at least one sensor 106, 108, 110, 112, and an output of the evaluation may be provided to the player 200 via the indicator 116.

In some examples, the musical instrument therapy device 100 further comprises a processing unit 114. For example, the processing unit 114 may operate to execute one or more program instructions that may include sufficient computer-executable instructions for performing one or more operations for providing throat muscle strengthening therapy or training as described herein.

In some examples, the musical instrument therapy device 100 may comprise a communication interface, such as a BLUETOOTH transmitter, configured to send information to an external device 124, such as to a speaker or an application on the player's mobile phone, to provide results/indications and/or audible output. In some examples, the application may provide a user interface (UI) including the indicator 116. In some examples, the musical instrument therapy device 100 further comprises a metronome 130 operative or configured to provide an audible and/or visual indication at a regular interval that can be set by a user/player.

The tubular body 102 defines an inner space, referred to herein as a bore 120, within which an air column may be formed. A first end and a second end of the tubular body 102 are open. In some examples, the tubular body 102 and the bore 120 may increase in cross-section from the first end to the second end. The tubular body 102 may be generally straight as illustrated or curved.

The mouthpiece 104 is located at the first end of the tubular body 102 and is configured for contact with a player's lips. According to an example, the mouthpiece 104 is designed to emulate the mouthpiece of a drone instrument. For example, the player 200 may place his/her lips on the mouthpiece 104 of the musical instrument therapy device 100 and, with loosely vibrating lips, blow air into the bore 120 (referred to herein as a blow input), forming an air column that may travel through the bore 120. In some examples, the mouthpiece 104 is formed integrally with the tubular body 102. In other examples, the mouthpiece 104 is attached to the tubular body 102.

In some examples, one or more auxiliary mouthpieces 126 may be provided, which can be selectively attached to the musical instrument therapy device 100. For example, an auxiliary mouthpiece 126 may correspond with another type of wind instrument (e.g., trumpet, saxophone, French horn, clarinet) and may be formed to simulate or otherwise emulate the mouthpiece of the corresponding wind instrument. The auxiliary mouthpieces 126 can be interchanged to provide training or therapy in association with the corresponding wind instrument. The auxiliary mouthpiece 126 may be configured to replace or attach to the (primary) mouthpiece 104.

The mute 118 may vary in design/function. In one example aspect, the mute 118 may be located at the second end of the tubular body 102, and may be attached in such a manner as to close the bore on the second end. The mute 118 may be configured to receive the air column and dampen or mute the drone sound of a typical drone instrument. Although illustrated as contained within the tubular body 102, in some examples, the mute 118 may extend outward through the opening at the second end of the tubular body 102. In another example aspect, the mute 118 may be embodied as a noise cancelling mechanism that is configured to electronically cancel sounds produced by blowing into the musical instrument therapy device 100.

According to one example, at least one sensor included in the sensor system may be included in the mouthpiece 104 and/or auxiliary mouthpiece 126. In some examples, the auxiliary mouthpiece 126 may be configured to attach to the musical instrument therapy device 100 such that the one or more sensors included in the auxiliary mouthpiece 126 are in communication with the processing unit 114. In some example aspects, the at least one sensor included in the mouthpiece 104 and/or auxiliary mouthpiece 126 may include one or more lip pressure sensors 106. The lip pressure sensors 106 may be positioned along the surface of the mouthpiece 104 and/or auxiliary mouthpiece 126 and may be configured to detect, measure, and report pressure, such as the force of the player's lips against the mouthpiece/musical instrument therapy device 100. According to one example aspect, one or more lip pressure sensors 106 may be configured to measure and report the force of the player's upper lips, and one or more other lip pressure sensors 106 may be configured to measure and report the force of the player's lower lips. In some examples, the lip pressure sensors 106 may be piezoresistive sensors.

In some examples, the sensor system may further one or more lip vibration sensors 108 included in the mouthpiece 104 and/or auxiliary mouthpiece 126. The lip vibration sensors 108 may be configured to sense and report vibration measurements of the player's lips when providing a blow input into the musical instrument therapy device 100. Lip vibration may be sensed via a pressure sensor, light sensor, accelerometer, or other type of vibration detection means.

In some examples, the sensor system may comprise an air pressure sensor 110. The air pressure sensor 110 may be positioned within the mouthpiece 104, auxiliary mouthpiece 126, or bore 120 and may be configured to measure and report the air pressure of the air column produced when the player 200 provides a blow input into the mouthpiece 104 or auxiliary mouthpiece 126 of the musical instrument therapy device 100. In some examples, the air pressure sensor 110 is embodied as a pressure gauge that may be able to provide an output reading of an air pressure value. For example, the output reading may be in the form of an analog display using a needle and a graduated scale or in the form of a digital display of the air pressure reading.

In some examples, the sensor system may comprise a frequency sensor 112. The frequency sensor 112 may be positioned within the mouthpiece 104, auxiliary mouthpiece 126, or bore 120 and may be configured to measure and report the frequency of the air column produced when the player 200 blows into the mouthpiece 104 or auxiliary mouthpiece 126 of the musical instrument therapy device 100. As one example, the player may be enabled to alter frequencies of the air column by altering the shape of his/her mouth, tongue movements, etc. In some examples, additional or alternative sensors (including a microphone) may be included in the sensor system.

In some examples, one or more of the sensors included in the sensor system may be in communication with or include an indicator 116. For example, one or more sensors may provide detected measurements associated with a blow input (e.g., air pressure, frequency) to the indicator 116, which may operate to indicate the detected measurements via a display, dial, and/or one or more lights. For example, according to one example implementation, the musical instrument therapy device 100 may operate as a mechanical device.

In other example implementations, the lip pressure sensors 106, lip vibration sensor 108, air pressure sensor 110, frequency sensor 112, and other sensors may be connected by a cable to the processing unit 114 and may be configured to report detecting signals and/or measurements associated with the player's lips and/or blow input to the processing unit 114. The processing unit 114 may include at least one processor communicatively connected to a memory, various wireless or wired communication interfaces (e.g., a Bluetooth interface, a USB port), and a battery. The processing unit 114 may further include or be in communication with a timing component 128 configured to measure and report time in association with other sensor data (e.g., a duration/frequency of a blow input, a duration/frequency of therapy or training). The processing unit 114 may be located within the tubular body 102 and is capable of operating the musical instrument therapy device 100 as described below. Such operating can be based on a set of preprogramed instructions (i.e., firmware) stored in the memory.

In some examples, the processing unit 114 may provide usage data and/or detected signals and measurements associated with the player's lips and/or blow input to an external device 124 (e.g., a mobile phone, a computing device) via a communication interface. For example, the external device 124 may include an application that may operate to receive the usage and/or signal data to compare against other data. In one example, a rate of usage of the musical instrument therapy device 100 may be compared against a rate of usage of another type of device, such as a CPAP device or other therapy or training device.

In some examples, the processing unit 114 can receive data from an external device 124 (e.g., a mobile phone, a computing device) via a communication interface. The processing unit 114 may store data received from an external device 124 in memory. For example, data received from an external device 124 may include baseline numbers, averages, norms, etc. The processing unit 114 may use data received from an external device 124 to compare against signals received from the various sensors 106, 108, 110, 112.

In some examples, the processing unit 114 can process signals received from the lip pressure sensors 106 to determine whether the force of the player's lips against the musical instrument therapy device 100 meet a predetermined threshold. In some examples, the predetermined threshold may be associated with a properly performed blow by an experienced player 200 that produces a typical drone via a typical drone instrument. As another example, the musical instrument therapy device 100 may be utilized with an auxiliary mouthpiece 126 embodied as a trumpet mouthpiece, and the predetermined threshold may be associated with a properly performed blow by an experienced trumpet player that produces a typical trumpet sound via a typical trumpet.

In some examples, the processing unit 114 can process signals received from the lip vibration sensor 108 to determine a relative pitch of the sound that is projected into and through the bore 120 of the musical instrument therapy device 100. For example, the player's mouth, lips, cheeks, tongue, throat, nose, vocal chords, diaphragm and lungs may all used to produce the sound, wherein looser lips and a lower lip vibration measurement may correspond with a lower pitch sound and tighter lips and a higher lip vibration measurement may correspond with a higher pitch sound.

In some examples, the processing unit 114 can process signals received from the air pressure sensor 110 to determine a length of a player's blow input and relative loudness.

In some examples, the processing unit 114 can process signals received from the frequency sensor 112 to determine the frequency of the sound that is projected into and through the bore 120 of the musical instrument therapy device 100. The processing unit 114 may determine the relation between the blow input frequency to notes of the chromatic scale, i.e., C, C#, D, D#, etc., and/or may generate a plot using signals received from the frequency sensor 112.

The processing unit 114 may perform one or more comparisons for determining and providing an output. One example output is a visual indication of measurements associated with a blow input. For example, the visual indication may be provided via the indicator 116. The indicator 116 may include a display, dial, and/or one or more lights that may be illuminated to indicate a measurement associated with a blow input. One example visual indication may provide a dynamic display that changes corresponding to the air pressure, the frequency, the player's lip vibration, and/or the player's lip pressure, etc.

Another example output is an indication that measurements associated with a blow input are within one or more predetermined thresholds associated with a properly performed blow input. According to one example aspect, a properly performed blow input may be associated with measurements that correspond to a particular blowing technique of a drone instrument or of another wind instrument. One example blowing technique is circular breathing, where a continuous blow input may be provided. Another example blowing technique is double-tonguing on a trumpet. Measurements associated with the predetermined thresholds may be stored in memory on the musical instrument therapy device 100 and/or in an application executing on an external device 124 with which the musical instrument therapy device 100 may be in communication. Time data provided by the timing mechanism 128 may be stored in association with the measurements.

According to another example aspect, a properly performed blow input may be associated with measurements that correspond to the tempo of blow inputs in relation to a beat provided by the metronome 130.

According to another example aspect, a properly performed blow input may be associated with measurements that correspond to strengthening of the pharyngeal airway muscles. As an example, with the aid of a healthcare professional, baseline measurements associated with the collapsibility of the player's airway may be collected (e.g., by utilizing an acoustic reflection measurement device to collect measurements while the player performs a modified Mueller's collapse). The baseline measurements may be communicated to and stored in memory on the musical instrument therapy device 100 and/or communicated to an application executing on an external device 124 with which the musical instrument therapy device 100 may be in communication. The player may then use the musical instrument therapy device 100 over a period of time (e.g., 30 minutes a day for 1 month) to strengthen his/her pharyngeal airway muscles (e.g., palatopharyngeus and stylohyoid), and then obtain a follow-up measurement of the collapsibility of the airway to track progress. Progress may be tracked by the musical instrument therapy device 100 and/or the application. For example, the player's measurements and other usage data may be compared against measurements and usage data obtained in relation to utilizing other therapy or training devices. As mentioned above, the player may wish to strengthen the pharyngeal airway muscles to help to maintain an open airway during sleep and prevent airway blockages, thereby reducing the chance of obstructed breathing during sleep. For example, strengthening and firming the muscles of the airway can help them to collapse less when the player is atonic or paralyzed during REM sleep. Moreover, using the musical instrument therapy device 100 may additionally strengthen the player's airway muscles for playing other wind instruments and/or provide a mechanism by which the player can practice blowing techniques for other types of wind instruments. Thus, adherence and effectiveness of using the musical instrument therapy device 100 in association with strengthening of the throat, cheek, tongue, and other related muscles and tissue due to use of the musical instrument therapy device 100 may be determined and tracked.

In some examples, the indicator 116 may include a display and/or one or more lights that may be illuminated to indicate when a properly performed blow input is executed by the player based on one or more sensor signals. As one example, a green light included in the indicator 116 may be illuminated when a determination is made that a blow input is within a threshold of a properly performed blow input. As another example, an amber light included in the indicator 116 may be illuminated with a determination is made that a blow input is approaching/near the threshold of a properly performed blow input.

Another example output may include an indication of the relation of a blow input to a note of the chromatic scale. For example, the indicator 116 may include a display and/or one or more lights that may be illuminated to indicate when a blow input corresponds with a particular note of the chromatic scale.

Another example output may include a visual indication of the length and/or relative loudness of the player's blow input based on signals received from the air pressure sensor 110.

In some examples, the musical instrument therapy device 100 may be configured to communicate with an external device 124, such as an application executing on the player's mobile computing device, to provide measurements collected from the various sensors 106, 108, 110, 112 that may be tracked by the application.

In some examples, the metronome 130 can additionally be used as a breathing exercise tool for the player. For example, the player can perform breathing exercises that align his/her breathing with the metronome beat, which can relax the state of the player's body and mind and help the player to better adapt to falling and staying asleep.

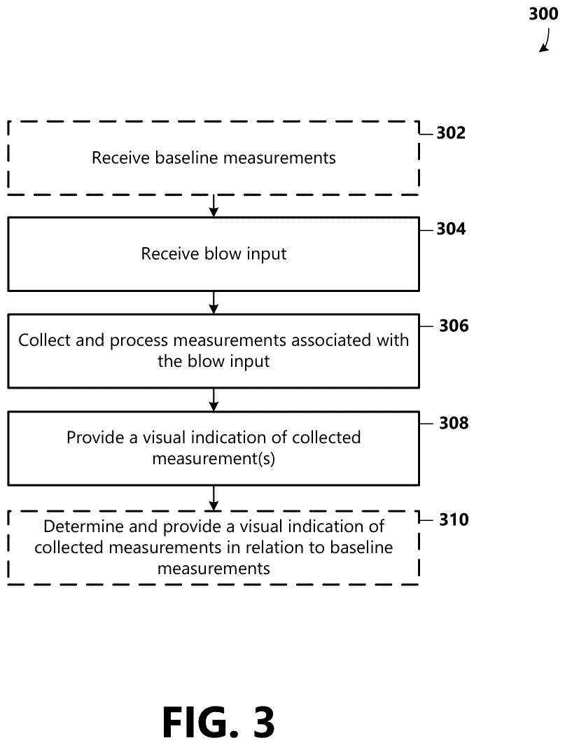

FIG. 3 is a flow diagram depicting general stages of an example process for providing throat muscle strengthening therapy according to an embodiment. With reference now to FIG. 3, at optional OPERATION 302, baseline measurements associated with the collapsibility of a player's 200 airway may be collected (e.g., by utilizing an acoustic reflection measurement device to collect measurements while the player performs a modified Mueller's collapse). The baseline measurements may be communicated to and stored in memory on the musical instrument therapy device 100 and/or communicated to an application executing on an external device 124 with which the musical instrument therapy device 100 may additionally be in communication.

At OPERATION 304, blow input may be received from the player 200. For example, the player 200 may place his/her lips on the mouthpiece 104 or an auxiliary mouthpiece 126 of the musical instrument therapy device 100 and, with loosely vibrating lips, provide a blow input into the bore 120, forming an air column that may travel through the bore 120 and be muted by the mute 118.

At OPERATION 306, various data may be collected by one or more of the various sensors 106, 108, 110, 112 operating on the therapy device 100. In some examples, the various data may be communicated to the processing unit 114, and processed by the processing unit 114. For example, various determinations may be made in association with the blow input.

At OPERATION 308, a visual indication associated with one or more of the collected sensor data may be provided via the indicator 116. For example, a visual or audible indicator 116 may indicate a properly performed blow input related to one or a combination of lip pressure, lip vibration, air pressure, frequency/pitch, time, etc.

At optional OPERATION 310, collected measurements may be evaluated based on the previously obtained baseline measurements, determinations may be made based on the baseline measurements, and visual indication associated with one or more of the collected sensor data in relation to the baseline measurements may be provided via the indicator 116 and displayed or otherwise presented on a computing device (i.e., external device 124) to the player 200.

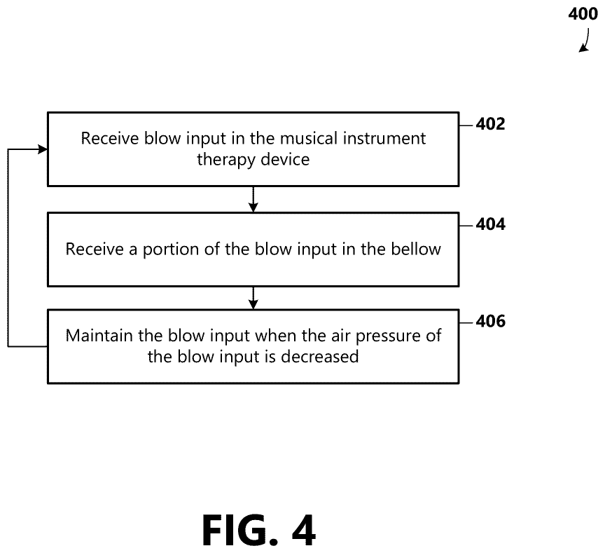

FIG. 4 is a flow diagram depicting general stages of an example process 400 for providing circular breathing support according to an embodiment. At OPERATION 402, a blow input may be received from the player 200. For example, the player 200 may place his/her lips on the mouthpiece 104 or an auxiliary mouthpiece 126 of the musical instrument therapy device 100 and provide a blow input into the bore 120, forming an air column that may travel through the bore 120 and that may be muted by the mute 118. In some examples, as the player 200 is providing the blow input, the player 200 may fill his/her/their mouth with air by puffing his/her/their cheeks with air from the lungs.

At OPERATION 404, a portion of the blow input may be received by the bellow 132.

At OPERATION 406, when the air pressure of the received blow input is less than the spring-force exerted by the spring 136, the spring 136 may move the plunger 134 and push air through the musical instrument therapy device 100. For example, the air pressure of the blow input may decrease as the player 200 attempts to tighten muscles in the throat to separate the mouth air from the lung air, and quickly inhale air into the lungs through the nose to replenish the lungs while continuing the blow input by expelling the air in the mouth by using the cheek muscles. The air pressure of the blow input may be maintained by the bellow 132 while the player 200 takes a breath in to repeat the circular breathing process 202. Thus, the bellow 132 may assist the player 200 in learning how to perform the circular breathing technique 202.

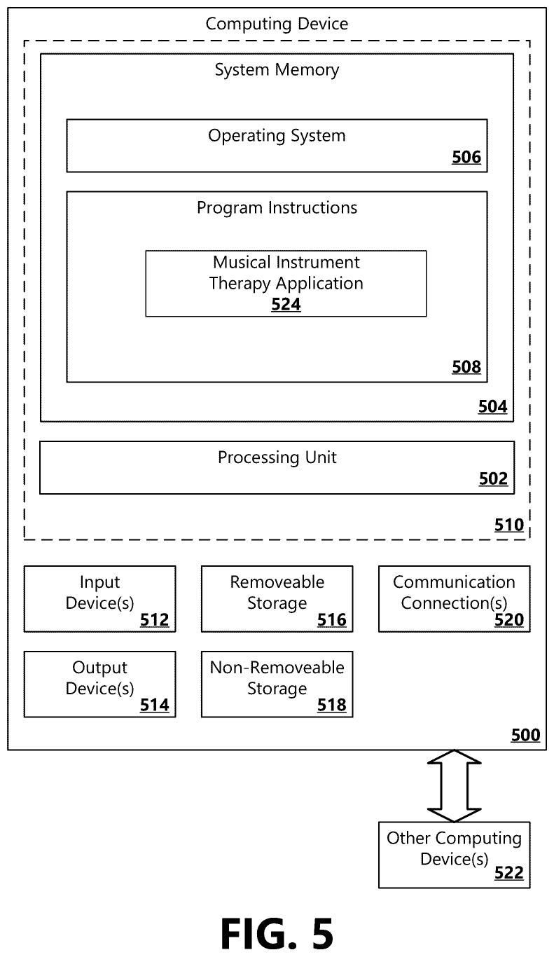

FIG. 5 is a block diagram illustrating physical components of an example computing device with which aspects may be practiced. The computing device 500 may include at least one processing unit 502 and a system memory 504. The system memory 504 may comprise, but is not limited to, volatile (e.g. random access memory (RAM)), non-volatile (e.g. read-only memory (ROM)), flash memory, or any combination thereof. System memory 504 may include operating system 506, one or more program instructions 508, and may include sufficient computer-executable instructions, which when executed, perform functionalities as described herein. In one example, the one or more program instructions 508 may include a musical instrument therapy application 524, which when executed, perform various functionalities as described herein. Operating system 506, for example, may be suitable for controlling the operation of computing device 500. Furthermore, aspects may be practiced in conjunction with a graphics library, other operating systems, or any other application program and is not limited to any particular application or system. This basic configuration is illustrated by those components within a dashed line 510. Computing device 500 may also include one or more input device(s) 512 (keyboard, mouse, pen, touch input device, etc.) and one or more output device(s) 514 (e.g., display, speakers, a printer, etc.).

The computing device 500 may also include additional data storage devices (removable or non-removable) such as, for example, magnetic disks, optical disks, or tape. Such additional storage is illustrated by a removable storage 516 and a non-removable storage 518. Computing device 500 may also contain a communication connection 520 that may allow computing device 500 to communicate with other computing devices 522, such as over a network in a distributed computing environment, for example, an intranet or the Internet. Communication connection 520 is one example of a communication medium, via which computer-readable transmission media (i.e., signals) may be propagated.

Programming modules may include routines, programs, components, data structures, and other types of structures that may perform particular tasks or that may implement particular abstract data types. Moreover, aspects may be practiced with other computer system configurations, including hand-held devices, multiprocessor systems, microprocessor-based or programmable user electronics, minicomputers, mainframe computers, and the like. Aspects may also be practiced in distributed computing environments where tasks are performed by remote processing devices that are linked through a communications network. In a distributed computing environment, programming modules may be located in both local and remote memory storage devices.

Furthermore, aspects may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (SoC)). Aspects may also be practiced using other technologies capable of performing logical operations such as, for example, AND, OR, and NOT, including, but not limited to, mechanical, optical, fluidic, and quantum technologies. In addition, aspects may be practiced within a general purpose computer or in any other circuits or systems.

Aspects may be implemented as a computer process (method), a computing system, or as an article of manufacture, such as a computer program product or computer-readable storage medium. The computer program product may be a computer storage medium readable by a computer system and encoding a computer program of instructions for executing a computer process. Accordingly, hardware or software (including firmware, resident software, micro-code, etc.) may provide aspects discussed herein. Aspects may take the form of a computer program product on a computer-usable or computer-readable storage medium having computer-usable or computer-readable program code embodied in the medium for use by, or in connection with, an instruction execution system.

Although aspects have been described as being associated with data stored in memory and other storage mediums, data can also be stored on or read from other types of computer-readable media, such as secondary storage devices, like hard disks, floppy disks, flash drives, or a CD-ROM, or other forms of RAM or ROM. The term computer-readable storage medium refers only to nontransitory devices and articles of manufacture that store data or computer-executable instructions readable by a computing device. The term computer-readable storage media does not include computer-readable transmission media.

Aspects of the present invention may be used in various distributed computing environments where tasks are performed by remote processing devices that are linked through a communications network.

Aspects of the invention may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 500 or any other computing devices 522, in combination with computing device 500, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described aspects.

The description and illustration of one or more aspects provided in this application are intended to provide a thorough and complete disclosure of the full scope of the subject matter to those skilled in the art and are not intended to limit or restrict the scope of the invention as claimed in any way. The aspects, examples, and details provided in this application are considered sufficient to convey possession and enable those skilled in the art to practice the best mode of the claimed invention. Descriptions of structures, resources, operations, and acts considered well-known to those skilled in the art may be brief or omitted to avoid obscuring lesser known or unique aspects of the subject matter of this application. The claimed invention should not be construed as being limited to any embodiment, aspects, example, or detail provided in this application unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently. Having been provided with the description and illustration of the present application, one skilled in the art may envision variations, modifications, and alternate embodiments falling within the spirit of the broader aspects of the general inventive concept provided in this application that do not depart from the broader scope of the present disclosure.

We claim:

1. A musical instrument therapy device, comprising:
a tubular body defining an air column;
a mouthpiece located at a first end of the tubular body configured to receive a blow input;
a mute located at a second end of the tubular body configured to mute the blow input;
at least one sensor configured to obtain measurements associated with the blow input; and
an indicator configured to provide an indication of the measurements.

2. The musical instrument therapy device of claim 1, further comprising a backpressure regulator operative to adjust an amount of back pressure in the tubular body.

3. The musical instrument therapy device of claim 1, further comprising a bellow operative to:
receive a portion of the blow input; and
when an air pressure of the blow input falls below a specified amount, maintain the blow input by pushing the portion of the blow input into tubular body.

4. The musical instrument therapy device of claim 1, further comprising a processing unit operative to provide the measurements collected from the at least one sensor to an external device to be tracked by an application executing on the external device.

5. The musical instrument therapy device of claim 1, further comprising a processing unit operative to:
make one or more determinations associated with the blow input based on the obtained measurements;
communicate the one or more determinations to the indicator for providing an indication of the one or more determinations.

6. The musical instrument therapy device of claim 5, wherein the one or more determinations includes a determination as to whether the blow input is within a threshold of a properly performed blow input based on one or more sensor measurements.

7. The musical instrument therapy device of claim 6, wherein the threshold corresponds with a note of a chromatic scale.

8. The musical instrument therapy device of claim 6, wherein:
the one or more sensor measurements includes air pressure sensor measurements; and
the threshold corresponds with a length and relative loudness of the blow input.

9. The musical instrument therapy device of claim 6, wherein the threshold corresponds with a circular breathing technique.

10. The musical instrument therapy device of claim 1, wherein:
the at least one sensor is an air pressure sensor; and
the indicator is an air pressure gauge.

11. The musical instrument therapy device of claim 1, wherein the mouthpiece is designed to emulate a mouthpiece of a drone instrument.

12. The musical instrument therapy device of claim 1, further comprising at least one auxiliary mouthpiece designed to emulate a mouthpiece of a wind instrument.

13. A system comprising:
at least one processing device;
a memory that stores computer executable instructions for providing strengthening therapy, which when executed by the at least processing device, operates to:
receive, from a musical instrument therapy device, measurements obtained by at least one sensor in the musical instrument therapy device, wherein the measurements are associated with a blow input received by the musical instrument therapy device; and
provide an indication of the received measurements.

14. The system of claim 13, wherein the system is further operative to track the received measurements.

15. The system of claim 13, wherein the system is further operative to:
receive a baseline measurement;
compare the received measurements against the baseline measurement; and
provide a visual indication of the received measurements in relation to the baseline measurement.

16. The system of claim 13, wherein the received measurements include at least one of:
lip pressure sensor measurements;
lip vibration sensor measurements,
air pressure sensor measurements; and
frequency sensor measurements.

17. A method for providing strengthening therapy comprising:
receiving a blow input via a mouthpiece of a musical instrument therapy device;
obtaining one or more measurements associated with the blow input from at least one sensor; and
providing an indication of the one or more measurements.

18. The method of claim 17, further comprising:
receiving a portion of the blow input in a bellow;
when an air pressure of the blow input received via the mouthpiece falls below a specified amount, maintaining the blow input by pushing the portion of the blow input through the musical instrument therapy device.

19. The method of claim 17, further comprising:
providing the one or more measurements to an external device to be tracked by an application executing on the external device.

20. The method of claim 17, further comprising:
making one or more determinations associated with the blow input based on the obtained measurements, wherein the one or more determinations includes a determination as to whether the blow input is within a threshold of a properly performed blow input based on one or more sensor measurements; and
communicating the one or more determinations to the indicator for providing an indication of the one or more determinations.
